(19) United States
(12) Patent Application Publication
(10) Pub. No.: US 2018/0180568 A1
(43) Pub. Date: Jun. 28, 2018

(54) METHOD MANUFACTURING SOLID ELECTROLYTE TYPE CARBON DIOXIDE SENSORS BY USING CO-SINTERING

(71) Applicants: Hyundai Motor Company, Seoul (KR); Kia Motors Corporation, Seoul (KR)

(72) Inventors: Jong-Min KWON, Gunpo-si (KR); Sun-Mi OH, Bucheon-si (KR); Hyun-Soo SOHN, Suwon-si (KR); Yang-Ki KIM, Seoul (KR); Tae-Won LEE, Seoul (KR); Ji-Hye KIM, Seoul (KR)

(73) Assignees: Hyundai Motor Company, Seoul (KR); Kia Motors Corporation, Seoul (KR)

(21) Appl. No.: 15/646,960

(22) Filed: Jul. 11, 2017

(30) Foreign Application Priority Data
Dec. 26, 2016 (KR) 10-2016-0179113

(51) Int. Cl. G01N 27/407; G01N 33/00

(52) U.S. Cl. CPC G01N 27/4074; G01N 27/4071; G01N 33/004

(57) ABSTRACT

A method for manufacturing the solid electrolyte type CO₂ sensor may include a bonding step of bonding a reference electrode on one surface of a solid electrolyte; a first stacking step of stacking a sensing electrode on the other surface of the solid electrolyte facing the surface bonded to the reference electrode; and a second stacking step of stacking a substrate on the other surface of the reference electrode facing the surface bonded to the solid electrolyte.

### CROSS-REFERENCE TO RELATED APPLICATIONS

The present application claims priority to Korean Patent Application No. 10-2016-0179113, filed on Dec. 26, 2016, the entire contents of which is incorporated herein for all purposes by this reference.

### BACKGROUND OF THE INVENTION

### Field of the Invention

The present invention relates to a method for manufacturing a solid electrolyte type CO₂ sensor. More particularly, it relates to a method for manufacturing a solid electrolyte type CO₂ sensor in which an initial stabilization speed of the sensor is increased, a manufacturing process is simplified, and accuracy of the sensor is improved by manufacturing the solid electrolyte type CO₂ sensor through co-sintering between a reference electrode and an electrolyte at predetermined time and temperature.

### Description of the Related Art

Vehicles are necessary transport means for the life of the human and make the life better. Recently, various optional specifications have been added with the upgrading of vehicles, and technologies for building a safe driving system of vehicles are being studied. As efforts to create a pleasant environment during operation are accelerated for safe driving, alternatives such as replacing components applied to the seat of the vehicle with substances with low carcinogen emissions have been proposed, and among them, an interest in the air quality of the interior of the vehicle is increasing and people are paying attention to carbon dioxide (CO₂). The carbon dioxide (CO₂) has a variety of effects on the human in an enclosed space and reduces brain activity of the driver and causes drowsy driving as factors affecting the driving among them. Accordingly, a function of real-time monitoring of carbon dioxide concentration for safe driving of the vehicle and a function of monitoring and restraining an environment that causes drowsy driving in real time by interlocking with a vehicle air conditioning system have received attention. As a result, a sensor for measuring the CO₂ concentration is required and as the solution thereof, an optical (NDIR: non-dispersion infrared) sensor, a semiconductor type gas sensor, and a solid electrolyte type household or general-use sensor has been proposed.

CO₂ is a chemically very stable gas in the atmosphere and a gas that is difficult to measure the concentration of. The optical sensor is the most widely used as the sensor for detecting the CO₂. This method is a method of measuring an amount of CO₂ by absorbing light having a specific wavelength of an emitted laser by the CO₂ in the air and detecting the decreased amount of the intensity of the light. This device has advantages of excellent selectivity, quantitativeness and reproducibility, but there is a problem in that a closed space for measurement is required and a volume is large and a weight is large due to physical sizes of the components and the filters. In particular, a driving unit and a measuring device are very expensive, and the configuration of a processing unit for the control is complicated. Thus, the overall cost of the measuring equipment is increased and as a result, even though the uses are very various, the devices have not been widely used.

Another method for measuring the CO₂ concentration is semiconductor type gas sensor using a semiconductor compound such as SnO₂ or TiO₂. This method is based on a principle of measuring the concentration of gas through a change in resistance when gas particles are adsorbed on the surface of the semiconductor compound. In this case, there is an advantage in that a thin-film device type sensor can be manufactured, but since it is difficult to distinguish different kinds of gas particles to be absorbed, it is also difficult to use the sensor as a device for selecting and measuring only carbon dioxide because there is a disadvantage in that the gas selectivity is remarkably deteriorated.

There is a problem in that a conventional solid electrolyte type household or general-purpose sensor requires a long time to stabilize the initial reaction. However, since the reaction needs to be stabilized within a short time for application to the vehicle, there is a problem in that a sensor of the related art requiring a long time is not suitable. Furthermore, in the sensor in the related art, there is a problem in that the accuracy is deteriorated or multiple heat-treating steps are required and thus manufacturing cost and time are increased. Accordingly, development of the solid electrolyte CO₂ sensor capable of overcoming the limitation of the solid electrolyte type general-use sensor has been required.

The information disclosed in this Background of the Invention section is only for enhancement of understanding of the general background of the invention and should not be taken as an acknowledgement or any form of suggestion that this information forms the prior art already known to a person skilled in the art.

### SUMMARY OF THE INVENTION

Various aspects of the present invention are directed to providing a method for manufacturing a solid electrolyte type CO₂ sensor by using co-sintering in which a stabilization speed of the sensor is increased and a manufacturing process is simplified.

Other technical objects desired to be achieved in the present invention are not limited to the aforementioned objects, and other technical objects not described above will be apparent to those skilled in the art from the disclosure of the present invention.

An exemplary embodiment of the present invention provides a method for manufacturing a solid electrolyte type CO₂ sensor including: a bonding step of bonding a reference electrode on one surface of a solid electrolyte; a first stacking step of stacking a sensing electrode on the other surface of the solid electrolyte facing the surface bonded to the reference electrode; and a second stacking step of stacking a substrate on the other surface of the reference electrode facing the surface bonded to the solid electrolyte.

The method may further include a coating step of coating a paste of the reference electrode on one surface of the solid electrolyte, before the bonding step.

The coating step may use screen printing.

In the bonding step, the solid electrolyte and the reference electrode may be co-sintered. The temperature of co-sintering may be from about 1150° C. to about 1250° C.

The temperature of co-sintering may be from about 1250° C. to about 1350° C.

The co-sintering time may be at least about 8 hrs to about 24 hrs.

The paste of the reference electrode may include yttria-stabilized zirconia.

The yttria-stabilized zirconia may be coated with a thickness of more than 0 μm to about 30 μm or less.

In yet another further preferred embodiment, the sensing electrode may be any one of A₂CO₃(A=Li, Na), BCO₃(B═Ba, Ca, Sr) or a mixture thereof.

The solid electrolyte may be formed as a green sheet.

In a still further preferred embodiment, the solid electrolyte may be Na_{1+X}Zr₂Si_{X}P_{3-X}O₁₂ and 0<X<3.

The solid electrolyte may be Li_{3X}La_{2/3-X}TiO₃ and 0.06<X<0.16.

The solid electrolyte may be Li₂+_{2X}Zn_{1-X}GeO₄ and 0<X<1.

The solid electrolyte may be Li₃PO_{4-X}N_{X} and 0.1<X<0.5.

The substrate may be made of alumina or mullite.

The method may further include sealing a side of the reference electrode with a sealant.

The sealant may be Na glass, Li glass, or a ceramic sealant.

According to the method for manufacturing the solid electrolyte type CO₂ sensor of the present invention, it is possible to provide a method for manufacturing the solid electrolyte type CO₂ sensor by using co-sintering in which an initial stabilization speed of the sensor is increased, a manufacturing process is simplified, and accuracy of the sensor is improved.

### BRIEF DESCRIPTION OF THE DRAWINGS

FIG. 1 is a configuration diagram of a solid electrolyte type gas sensor in the related art.

FIG. 2 is a configuration diagram of a solid electrolyte type CO₂ sensor according to an exemplary embodiment of the present invention.

FIG. 3 is a configuration diagram for measuring a voltage of the solid electrolyte type CO₂ sensor according to the exemplary embodiment of the present invention.

FIG. 4 is a schematic diagram of a solid electrolyte type gas sensor in the related art.

FIG. 5 is a schematic diagram of a reaction unit of a reference electrode of the solid electrolyte type gas sensor in the related art.

FIG. 6 is a schematic diagram of the solid electrolyte type CO₂ sensor according to the exemplary embodiment of the present invention.

FIG. 7 is a schematic diagram of a reaction unit of a reference electrode of the solid electrolyte type CO₂ sensor according to the exemplary embodiment of the present invention.

FIG. 8 is a flowchart of a coating step and a bonding step for co-sintering according to the exemplary embodiment of the present invention.

FIG. 9 is an enlarged photograph of a co-sintered electrolyte and an YSZ layer as the reference electrode according to the exemplary embodiment of the present invention.

FIG. 10 is an enlarged photograph of an YSZ layer heat-treated on the sintered electrolyte through screen printing in the related art.

FIG. 11 is an enlarged photograph of the YSZ layer as the reference electrode which is co-sintered at 1130° C.

FIG. 12 is an enlarged photograph of an YSZ layer as the reference electrode which is co-sintered at 1180° C. according to the exemplary embodiment of the present invention.

FIG. 13 is an enlarged photograph of the YSZ layer as the reference electrode which is co-sintered for less than 8 hours.

FIG. 14 is a photograph of the YSZ layer as the reference electrode coated with a thickness of 50 μm.

FIG. 15 is a graph illustrating a measured voltage with time according to an Example of the present invention and a Comparative Example as the related art in the case where a solid electrolyte is NASICON.

FIG. 16 is a graph illustrating normalizing of a measured voltage with time according to an Example of the present invention and a Comparative Example as the related art in the case where a solid electrolyte is NASICON.

FIG. 17 is a graph illustrating a measured voltage with time according to an Example of the present invention and a Comparative Example as the related art in the case where a solid electrolyte is LLT.

FIG. 18 is a graph illustrating normalizing of a measured voltage with time according to an Example of the present invention and a Comparative Example as the related art in the case where a solid electrolyte is LLT.

FIG. 19 is a graph illustrating a changed voltage with time in the case of changing a concentration of carbon dioxide according to an exemplary embodiment of the present invention.

FIG. 20 is a graph illustrating a changed voltage according to a concentration of carbon dioxide according to an exemplary embodiment of the present invention and a Comparative Example as the related art.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION OF THE EMBODIMENTS

Hereinafter, exemplary embodiments of the present invention will be described in detail with reference to the accompanying drawings. Terms or words used in the present specification and claims, which will be described below should not be interpreted as being limited to typical or dictionary meanings, but should be interpreted as having meanings and concepts which comply with the technical spirit of the present invention, based on the principle that an inventor can appropriately define the concept of the term to describe his/her own invention in the best manner. Therefore, configurations illustrated in the embodiments and the drawings described in the present specification are only the most preferred embodiment of the present invention and do not represent all of the technical spirit of the present invention, and thus it is to be understood that various equivalents and modified examples, which may replace the configurations, are possible when filing the present application.

The present invention relates to a method for manufacturing a solid electrolyte type CO₂ sensor by using co-sintering and more particularly, to a method for manufacturing a solid electrolyte type CO₂ sensor by bonding a solid electrolyte and a reference electrode using co-sintering to improve an initial stabilization speed of the sensor and simplify a manufacturing process.

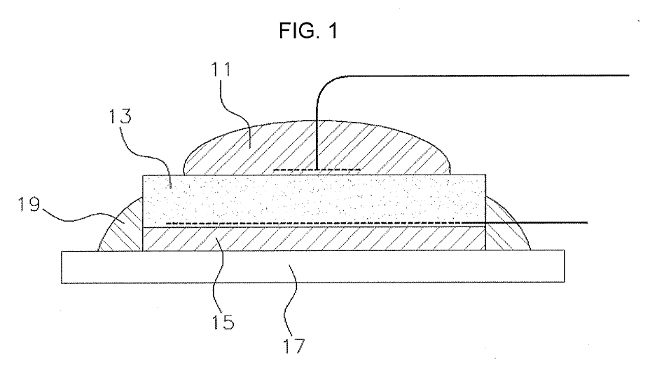

FIG. 1 is a configuration diagram of a solid electrolyte type gas sensor in the related art. When describing the related art in detail, as illustrated in FIG. 1, the solid electrolyte type gas sensor is configured by a structure in which a sensing electrode 11, a solid electrolyte 13, a reference electrode 15, and a substrate 17 are stacked in sequence and a side of the reference electrode is sealed by a sealant 19. The solid electrolyte type gas sensor in the related art uses a method in which in order to prepare a double layer configured by the solid electrolyte 13 and the reference electrode 15, after respective sintered bodies are formed, the sintered body of the solid electrolyte 13 and the sintered body of the reference electrode 15 are bonded to each other by using a deposition method, that is, a method such as PLD or sputtering or a thermal bonding method at about 950° C.

However, the deposition method in the related art has a limitation in mass production due to required time and cost. Furthermore, there is a problem in that the thermal deposition method in the related art may be applied to only a case where the area of the sensor is large, but may not be applied to manufacturing the sensor with a size applied to the vehicle of the present invention. Further, the thermal bonding method is a method of bonding the reference electrode and the solid electrolyte 13 which are respectively sintered, by applying pressure and heat, and there is a problem in that the reference electrode 15 and the solid electrolyte 13 are not uniformly bonded to each other and the sintered bodies are broken due to the heat and the pressure.

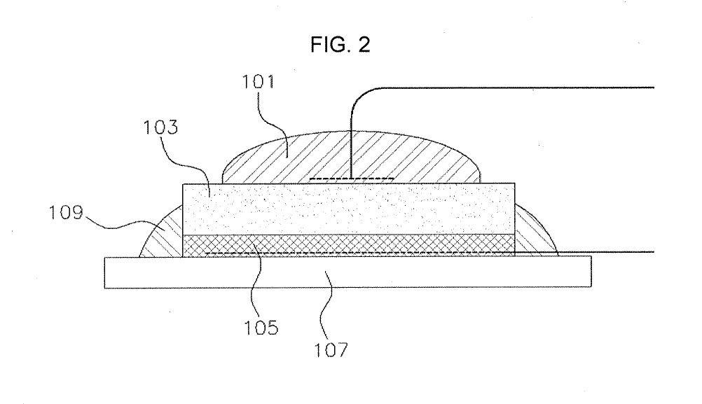

FIG. 2 is a configuration diagram of a solid electrolyte type CO₂ sensor according to an exemplary embodiment of the present invention. When describing the solid electrolyte type CO₂ sensor according to the exemplary embodiment of the present invention in detail, a sensing electrode 101, a solid electrolyte 103, a reference electrode 105 and a substrate 107 are stacked in sequence and the solid electrolyte and the reference electrode are bonded to each other by co-sintering. Further, in some cases, a structure which is sealed by a sealant 109 at the side of the reference electrode 105 may be included.

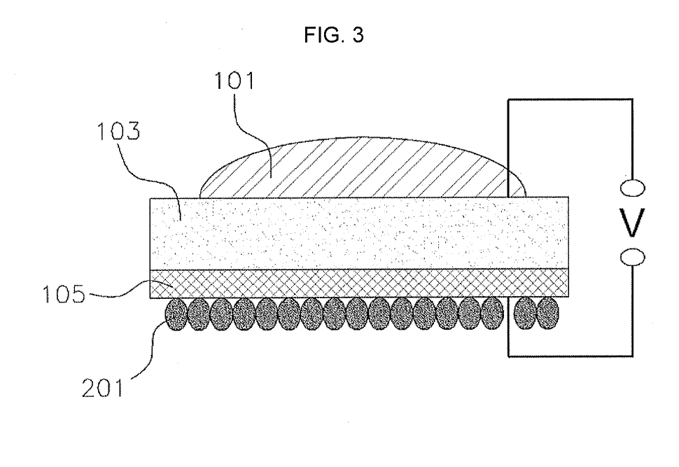

FIG. 3 is a configuration diagram for measuring a voltage of the solid electrolyte type CO₂ sensor according to the exemplary embodiment of the present invention. When in the solid electrolyte 103, NASICON is used as an electrolyte, a theoretical background of the solid electrolyte type CO₂ sensor of the present invention may be expressed as a galvanic cell represented by the following Chemical Formula 1.

$${CO}_{2} , {O}_{2} , \\ Pt \vert {Na}_{2} {{CO}_{3}}_{{{P}_{{O}_{z} : 1}}_{{a}_{Na : 1}}} ‖ NASIC {ON ‖}_{{{P}_{{O}_{z} : 2}}_{{a}_{Na : 2}}} YS {Z \vert {O}_{2}}_{{P}_{{O}_{z} : 3}} , Pt \qquad [ Chemical Formula 1 ]$$

In this case, since the CO₂ sensor is in the same gas atmosphere, P_{O}_{_{2,}}₁=P_{O}_{_{2,}}₃. Accordingly, a voltage applied to two terminals of the sensor may be expressed by Equation 1 below.

$$V = - \frac{RT}{F} ln \frac{{α}_{Na : 1}}{{α}_{Na : 2}} + \frac{RT}{4 F} ln \frac{{P}_{{O}_{z} : 2}}{{P}_{{O}_{z} : 3}} \qquad [ Equation 1 ]$$

In Equation 1, R is a gas constant and F is a Faraday constant. Herein, a reaction of the sensing electrode 101 and a reaction occurring in an interface between the solid electrolyte 103 and the reference electrode 105 are represented by Chemical Formulas 2 and 3 below.

Reaction of sensing electrode: Na₂CO₃->2Na⁺+2e⁻+½O₂+CO₂ [Chemical Formula 2]

Interface reaction between reference electrode and solid electrolyte: Na₂O->2Na⁺+O²⁻ [Chemical Formula 3]

Further, the reaction of the reference electrode 105 is represented by Chemical Formula 4.

Reaction of reference electrode: ½O₂+2e⁻->O²⁻ [Chemical Formula 4]

Further, according to the Chemical Formulas 2 and 3, the reaction of the sensing electrode 101 and the reaction occurring in an interface between the solid electrolyte 103 and the reference electrode 105 are represented by Equations 2 and 3 below when being expressed as a function of Gibbs free energy.

$$\begin{matrix}Δ {G}_{{CO}_{2}}^{f} - Δ {G}_{{Na}_{2} {CO}_{3}}^{f} = - RT ln \frac{{a}_{Na : 1}^{2} {P}_{{O}_{z} : 3}^{1 / 2} {P}_{{CO}_{z}}}{{a}_{{Na}_{2} {CO}_{3}}} & [ Equation 2 ] \\ - Δ {G}_{{Na}_{2} O}^{f} = - RT ln \frac{{a}_{Na : 2}^{2} {P}_{{O}_{z} : 2}^{1 / 2}}{{α}_{{Na}_{2} O}} & [ Equation 3 ]\end{matrix}$$

Meanwhile, the function of Gibbs free energy has a relation of Equation 4 below.

ΔG⁰=ΔG_{No}_{₂}_{O}^{f}+ΔG_{CO}_{₂}^{f}−ΔG_{No}_{₂}_{CO}_{₃} [Equation 4]

The Equations 2 and 3 are substituted to the Equation 1 to be Equation 5 below.

$$V = α - \frac{2.303 RT}{2 F} log {P}_{{CO}_{2}} \qquad [ Equation 5 ]$$

In Equation 5, α is represented by Equation 6 below.

$$α = - \frac{1}{2 F} ( RT ln \frac{{a}_{{Na}_{2} O}}{{a}_{{Na}_{2} {CO}_{3}}} + Δ {G}^{0} ) \qquad [ Equation 6 ]$$

From Equation 5, it can be seen that a voltage V measured from the solid electrolyte CO₂ sensor at a constant temperature is proportional to a log value of a CO₂ concentration. The precondition for such an interpretation is that the activity of Na₂O in a Na ion conductor, that is, the NASICON needs to be constant under a measurement condition. This may be described by a Gibbs phase rule. There are three kinds of metal oxides constituting the Na ion conductor (NASICON) verified as one phase and a ratio of cations is fixed in the electrolyte preparing step, and as a result, Equation 7 below is established under air pressure and thus the activity of all metal oxides constituting the Na ion conductor is a temperature-dependent function.

F (degree of freedom)=3 (number of atoms)−1 (number of phases)−2 (fixed cation ratio)+1 (temperature)=1 [Equation 7]

Accordingly, it can be seen that since the voltage of the sensor given by Equation 5 is represented by a function of CO₂ concentration at a constant temperature, the gas sensor of the present invention is suitable for the gas sensor for sensing CO₂.

Additionally, when in the solid electrolyte 105, LLT is used as an electrolyte, a theoretical background of the solid electrolyte type CO₂ sensor of the present invention may be expressed as a galvanic cell represented by the following Chemical Formula 5.

$${CO}_{2} , {O}_{2} , Pt \vert {Li}_{2} {CO}_{3} {‖ LLT}_{{{P}_{{O}_{z} : 1}}_{{a}_{Li : 1}}} {‖ YSZ}_{{{P}_{{O}_{z} : 2}}_{{a}_{Li : 2}}} {\vert {O}_{2} , Pt}_{{P}_{{O}_{z} : 3}} \qquad [ Chemical Formula 5 ]$$

In this case, since the CO₂ sensor is in the same gas atmosphere, P_{O}_{_{2,}}₁=P_{O}_{_{2,}}₃. Accordingly, a voltage applied to two terminals of the sensor may be expressed by Equation 8 below.

$$V = - \frac{RT}{F} ln \frac{{a}_{Li : 1}}{{a}_{Li : 2}} + \frac{RT}{4 F} ln \frac{{P}_{{O}_{z} : 2}}{{P}_{{O}_{z} : 3}} \qquad [ Equation 8 ]$$

In Equation 8, R is a gas constant and F is a Faraday constant. Herein, a reaction of the sensing electrode 101 and a reaction occurring in an interface between the solid electrolyte 103 and the reference electrode 105 are illustrated in Chemical Formulas 6 and 7 below.

Reaction of sensing electrode: Li₂CO₃->2Li⁺+2e⁻+½O₂+CO₂ [Chemical Formula 6]

Interface reaction between reference electrode and solid electrolyte: Li₂O->2Li⁺+O²⁻ [Chemical Formula 7]

Further, the reaction of the reference electrode 105 is represented by Chemical Formula 8.

Reaction of reference electrode: ½O₂+2e⁻->O²⁻ [Chemical Formula 8]

According to the Chemical Formulas 6 and 7, the reaction of the sensing electrode 101 and the reaction occurring in an interface between the solid electrolyte 103 and the reference electrode 105 are represented by Equations 9 and 10 below when being expressed as a function of Gibbs free energy.

$$\begin{matrix}Δ {G}_{{CO}_{2}}^{f} - Δ {G}_{{Li}_{2} {CO}_{3}}^{f} = - RT ln \frac{{a}_{Li : 1}^{2} {P}_{{O}_{z} : 3}^{1 / 2} {P}_{{CO}_{z}}}{{a}_{{Li}_{2} {CO}_{3}}} & [ Equation 9 ] \\ - Δ {G}_{{Li}_{2} O}^{f} = - RT ln \frac{{a}_{Li : 2}^{2} {P}_{{O}_{z} : 2}^{1 / 2}}{{a}_{{Li}_{2} O}} & [ Equation 10 ]\end{matrix}$$

Meanwhile, the function of Gibbs free energy has a relation of Equation 11 below.

ΔG⁰=ΔG_{Li}_{₂}_{O}^{f}+ΔG_{CO}_{₂}^{f}−ΔG_{Li}_{₂}_{CO}_{₃} [Equation 4]

The Equations 9 and 10 are substituted to the Equation 8 to be Equation 12 below.

$$V = α - \frac{2.303 RT}{2 F} log {P}_{{CO}_{2}} \qquad [ Equation 12 ]$$

In Equation 12, a is represented by Equation 13 below.

$$α = - \frac{1}{2 F} ( RT ln \frac{{a}_{{Li}_{2} O}}{{a}_{{Li}_{2} {CO}_{3}}} + Δ {G}^{0} ) \qquad [ Equation 13 ]$$

From Equation 12, it can be seen that a voltage V measured from the solid electrolyte CO₂ sensor at a constant temperature is proportional to a log value of a CO₂ concentration. The precondition for this interpretation is that the activity of Li₂O in a Li ion conductor needs to be constant under a measurement condition. This may be described by a Gibbs phase rule. There are three kinds of metal oxides constituting the Li ion conductor (LLT) verified as one phase and a ratio of cations is fixed in the electrolyte preparing step, and as a result, Equation 14 below is established under air pressure and thus the activity of all metal oxides constituting the Li ion conductor is a temperature-dependent function.

F (degree of freedom)=3 (number of atoms)−1 (number of phases)−2 (fixed cation ratio)+1 (temperature)=1 [Equation 14]

Accordingly, it can be seen that since the voltage of the sensor given by Equation 12 is represented by a function of CO₂ concentration at a constant temperature, the gas sensor of the present invention is suitable for the gas sensor for sensing CO₂.

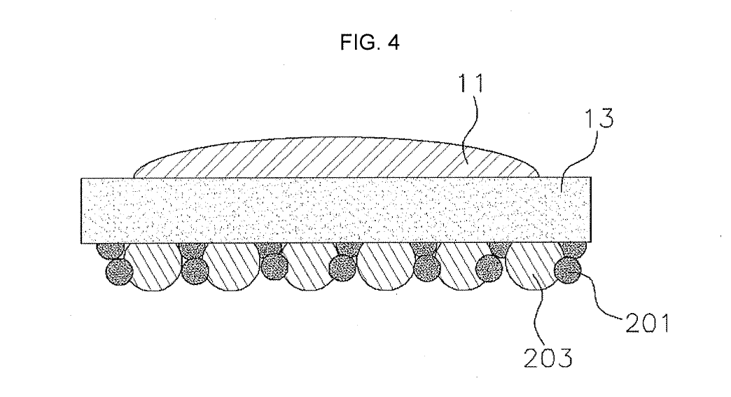

FIG. 4 is a schematic diagram of a solid electrolyte type gas sensor in the related art. The structure of the solid electrolyte type gas sensor includes carbonate and Au as the sensing electrode 11, NASICON or a Li ion conductor as the solid electrolyte 13, and a Li—Ti—O2 phase mixture 203 and Pt 201 as the reference electrode 15. The reaction between the sensing electrode 11 and the reference electrode 15 of the solid electrolyte type gas sensor in the related art is represented by Chemical Formulas 9 and 10 below.

Reaction of sensing electrode: Li₂CO₃->2Li⁺+2e⁻+½O₂+CO₂ [Chemical Formula 9]

Reaction of reference electrode: Li₂TiO₃->2Li⁺+2e⁻+TiO₂+½O₂ [Chemical Formula 10]

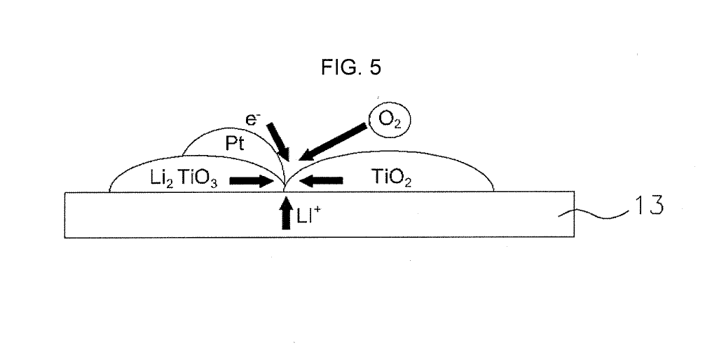

FIG. 5 is a schematic diagram of a reaction unit of a reference electrode of the solid electrolyte type gas sensor in the related art. As illustrated in FIG. 5, it can be seen that the electrode reaction occurs at a boundary where five phases of Pt, Li₂TiO₃, TiO₂, the surface of the NASICON or the surface of the Li ion conductor, and oxygen meet with each other at the same time (5 phase boundary). As a result, probability of the 5 phase boundary is low and thus there is a problem in that electrode resistance is large and a reaction in the electrode is slow. Furthermore, there is a problem in that the reference electrode has no layer for protecting the reference electrode to be exposed moisture, a volatile organic compound (VOC), or the like and thus the reaction of the reference electrode is disturbed.

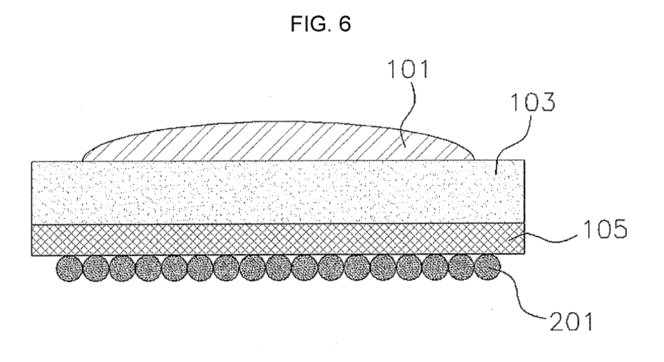

FIG. 6 is a schematic diagram of the solid electrolyte type CO₂ sensor according to the exemplary embodiment of the present invention. A reaction of the sensing electrode 101, a reaction of the reference electrode 105, and an interface reaction among the reference electrode 105 and the solid electrolyte 103 of the solid electrolyte type CO₂ sensor according to the exemplary embodiment of the present invention are represented by Chemical Formulas 11 to 13.

Reaction of sensing electrode: Li₂CO₃->2Li⁺+2e⁻+½O₂+CO₂ [Chemical Formula 11]

Reaction of reference electrode: ½O₂+2e⁻->O²⁻ [Chemical Formula 12]

Interface reaction between reference electrode and solid electrolyte: Li₂O->2Li⁺+O²⁻ [Chemical Formula 13]

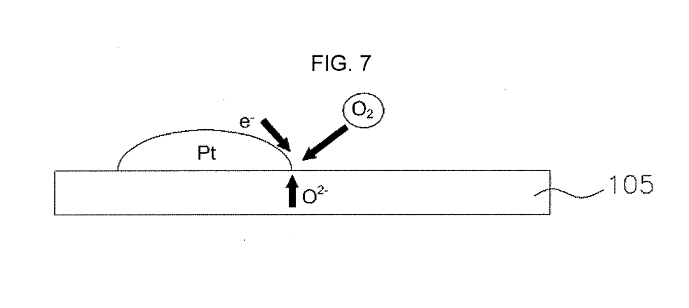

FIG. 7 is a schematic diagram of a reaction unit of a reference electrode of the solid electrolyte type CO₂ sensor according to the exemplary embodiment of the present invention. As illustrated in FIG. 7, it can be seen that the electrode reaction occurs at a boundary where three phases of Pt, the surface of the NASICON or the surface of the Li ion conductor, and oxygen meet with each other at the same time. As a result, it can be seen that the probability of existence of the 3 phase boundary of the present invention is much higher than that of the 5 phase boundary in the related art, and thus the electrode resistance is relatively small and the reaction speed is relatively high as compared with the related art. Further, the densely structured YSZ layer has an advantage of protecting the surface of the solid electrolyte which is vulnerable to moisture, VOC, or the like.

The present invention relates to a method for manufacturing a solid electrolyte type CO₂ sensor using co-sintering and more particularly, to a method for manufacturing a solid electrolyte type CO₂ sensor including a bonding step of bonding the reference electrode 105 on one surface of the solid electrolyte 103; a first stacking step of stacking the sensing electrode 101 on the other surface of the solid electrolyte 103 facing the surface bonded to the reference electrode 105; and a second stacking step of stacking the substrate 107 on the other surface of the reference electrode 105 facing the surface bonded to the solid electrolyte 103. In the present invention, before the bonding step, the method may further include a coating step of coating a paste of the reference electrode 105 on one surface of the solid electrolyte 103, and the coating step may use screen printing and the bonding step may include co-sintering the solid electrolyte 103 and the reference electrode 105. Further, the paste of the reference electrode 105 may include yttria-stabilized zirconia (YSZ).

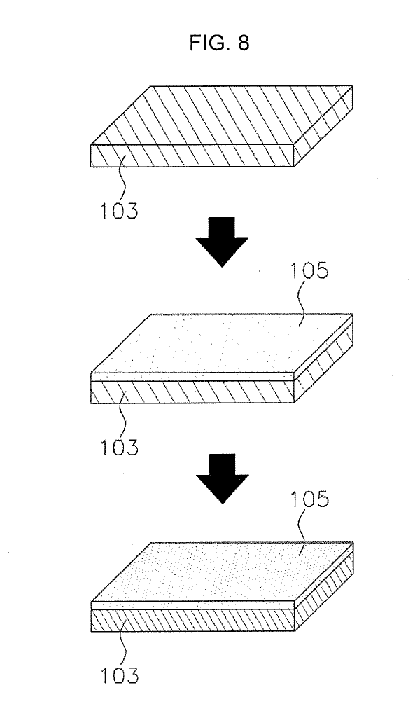

In the related art, the solid electrolyte was sintered at a sintering temperature and then the YSZ layer was sintered at about 1500° C. That is, the solid electrolyte and the YSZ layer were sintered at different temperatures, respectively, and then the solid electrolyte and the YSZ layer were thermally bonded to each other by using heat and pressure to form a double layer. However, in the bonding method, there are problems in that the solid electrolyte and the YSZ layer were not uniformly bonded and broken by heat and pressure and accuracy of the sensor was deteriorated. FIG. 8 is a flowchart of a coating step and a bonding step for co-sintering according to the exemplary embodiment of the present invention. The present invention used co-sintering by the method illustrated in FIG. 8. In detail, the solid electrolyte 103 was formed as a green sheet and then the YSZ paste as the reference electrode 105 was coated, that is, screen-printed on the green sheet of the solid electrolyte. Thereafter, the solid electrolyte 103 coated with the YSZ layer was co-sintered for about 8 to about 24 hours (e.g., about 8 hours, 9, 10, 11, 12, 13, 14, 15, 16, 17, 18, 19, 20, 21, 22, 23, or about 24 hours) at a temperature of from about 1150° C. to about 1250° C. (e.g., about 1150° C., 1152° C., 1154° C., 1156° C., 1158° C., 1160° C., 1162° C., 1164° C., 1166° C., 1168° C., 1170° C., 1172° C., 1174° C., 1176° C., 1178° C., 1180° C., 1182° C., 1184° C., 1186° C., 1188° C., 1190° C., 1192° C., 1194° C., 1196° C., 1198° C., 1200° C., 1202° C., 1204° C., 1206° C., 1208° C., 1210° C., 1212° C., 1214° C., 1216° C., 1218° C., 1220° C., 1222° C., 1224° C., 1226° C., 1228° C., 1230° C., 1232° C., 1234° C., 1236° C., 1238° C., 1240° C., 1242° C., 1244° C., 1246° C., 1248° C., or about 1250° C.) or a temperature of 1250° C. to 1350° C. (e.g., about 1250° C., 1252° C., 1254° C., 1256° C., 1258° C., 1260° C., 1262° C., 1264° C., 1266° C., 1268° C., 1270° C., 1272° C., 1274° C., 1276° C., 1278° C., 1280° C., 1282° C., 1284° C., 1286° C., 1288° C., 1290° C., 1292° C., 1294° C., 1296° C., 1298° C., 1300° C., 1302° C., 1304° C., 1306° C., 1308° C., 1310° C., 1312° C., 1314° C., 1316° C., 1318° C., 1320° C., 1322° C., 1324° C., 1326° C., 1328° C., 1330° C., 1332° C., 1334° C., 1336° C., 1338° C., 1340° C., 1342° C., 1344° C., 1346° C., 1348° C., or about 1350° C.). In more detail, when the solid electrolyte is the NASICON, the solid electrolyte may be co-sintered for about 8 to about 24 hours (e.g., about 8 hours, 9, 10, 11, 12, 13, 14, 15, 16, 17, 18, 19, 20, 21, 22, 23, or about 24 hours) at a temperature of from about 1150° C. to about 1250° C. (e.g., about 1150° C., 1152° C., 1154° C., 1156° C., 1158° C., 1160° C., 1162° C., 1164° C., 1166° C., 1168° C., 1170° C., 1172° C., 1174° C., 1176° C., 1178° C., 1180° C., 1182° C., 1184° C., 1186° C., 1188° C., 1190° C., 1192° C., 1194° C., 1196° C., 1198° C., 1200° C., 1202° C., 1204° C., 1206° C., 1208° C., 1210° C., 1212° C., 1214° C., 1216° C., 1218° C., 1220° C., 1222° C., 1224° C., 1226° C., 1228° C., 1230° C., 1232° C., 1234° C., 1236° C., 1238° C., 1240° C., 1242° C., 1244° C., 1246° C., 1248° C., or about 1250° C.), and when the solid electrolyte is the LLT, the solid electrolyte may be co-sintered for about 8 to about 24 hours (e.g., about 8 hours, 9, 10, 11, 12, 13, 14, 15, 16, 17, 18, 19, 20, 21, 22, 23, or about 24 hours) at a temperature of about 1250° C. to about 1350° C. (e.g., about 1250° C., 1252° C., 1254° C., 1256° C., 1258° C., 1260° C., 1262° C., 1264° C., 1266° C., 1268° C., 1270° C., 1272° C., 1274° C., 1276° C., 1278° C., 1280° C., 1282° C., 1284° C., 1286° C., 1288° C., 1290° C., 1292° C., 1294° C., 1296° C., 1298° C., 1300° C., 1302° C., 1304° C., 1306° C., 1308° C., 1310° C., 1312° C., 1314° C., 1316° C., 1318° C., 1320° C., 1322° C., 1324° C., 1326° C., 1328° C., 1330° C., 1332° C., 1334° C., 1336° C., 1338° C., 1340° C., 1342° C., 1344° C., 1346° C., 1348° C., or about 1350° C.).

According to the double layer of the solid electrolyte/YSZ layer formed by the manufacturing method of the present invention, as compared with the related art, there are advantages in that bonding between the two layers is uniformly formed and a triple heat-treating process is solved by one heat-treating process and thus the manufacturing cost is reduced, the manufacturing time is reduced, and the initial stabilization speed of the sensor is improved.

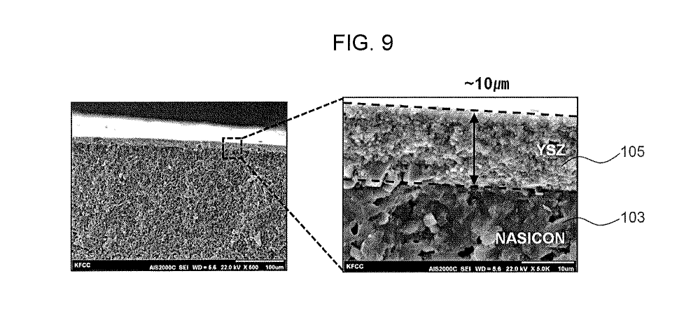

FIG. 9 is an enlarged photograph of a co-sintered double layer of the solid electrolyte 103 and the YSZ layer as the reference electrode 105 according to the exemplary embodiment of the present invention. The co-sintered double layer is sintered for about 8 hours at about 1280° C. after screen-printing the YSZ layer having a thickness of about 20 μm on the green sheet of the NASICON which is the solid electrolyte. As illustrated in FIG. 9, it can be seen that the YSZ layer is densely formed on the NASICON and the NASICON which is the solid electrolyte is sintered at a lower temperature than the sintering temperature of the YSZ by the sintered force.

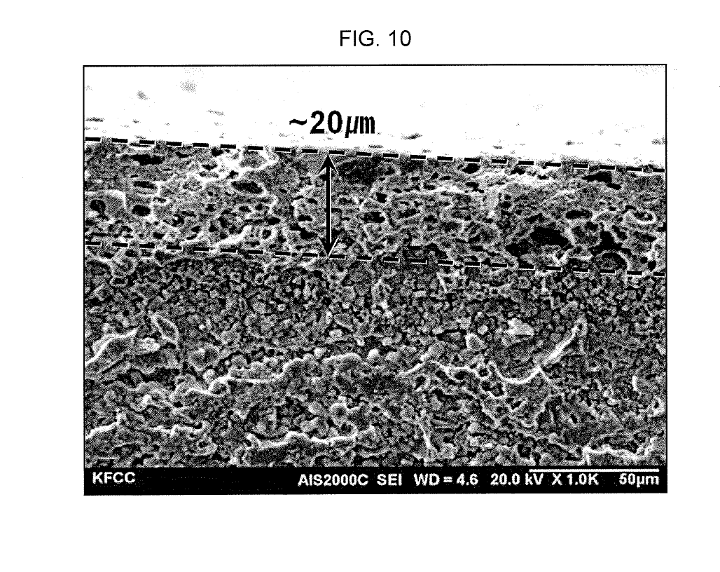

FIG. 10 is an enlarged photograph of a surface heat-treated by screen-printing the YSZ layer which is the reference electrode on the sintered electrolyte in the related art. In the related art, the solid electrolyte and the YSZ layer which is the reference electrode are thermally bonded to each other while being separately sintered. As a result, the YSZ layer may not receive the stress due to the shrinkage due to the sintering of the solid electrolyte, so that the YSZ layer is not densified at the temperature and time, and there is a problem that in order to densify the YSZ layer, the temperature needs to be increased to the YSZ sintering temperature.

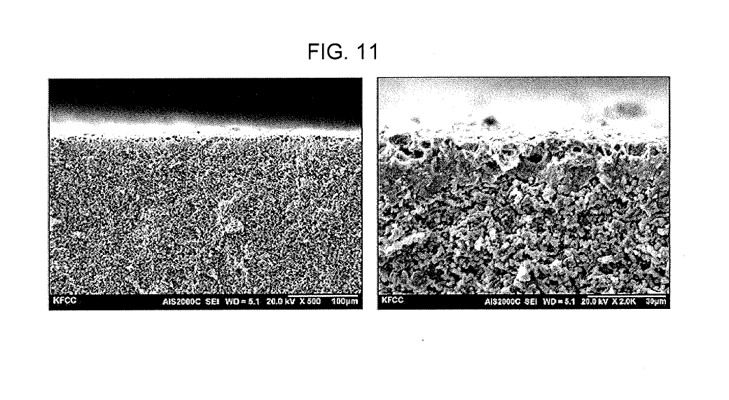

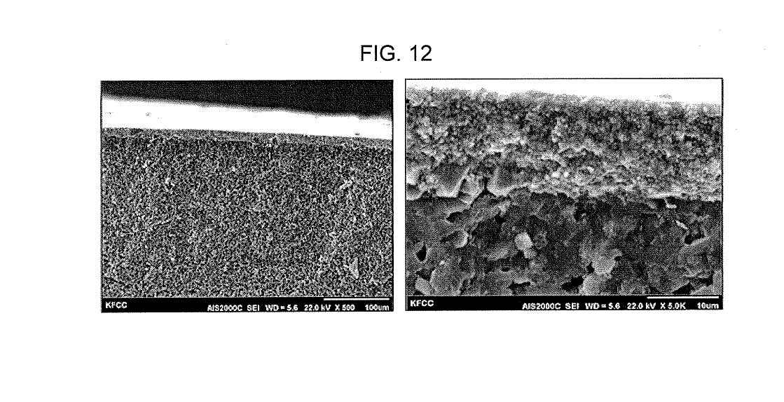

In the present invention, the temperature of co-sintering may be about 1150° C. to about 1250° C. (e.g., about 1150° C., 1152° C., 1154° C., 1156° C., 1158° C., 1160° C., 1162° C., 1164° C., 1166° C., 1168° C., 1170° C., 1172° C., 1174° C., 1176° C., 1178° C., 1180° C., 1182° C., 1184° C., 1186° C., 1188° C., 1190° C., 1192° C., 1194° C., 1196° C., 1198° C., 1200° C., 1202° C., 1204° C., 1206° C., 1208° C., 1210° C., 1212° C., 1214° C., 1216° C., 1218° C., 1220° C., 1222° C., 1224° C., 1226° C., 1228° C., 1230° C., 1232° C., 1234° C., 1236° C., 1238° C., 1240° C., 1242° C., 1244° C., 1246° C., 1248° C., or about 1250° C.). When the co-sintering temperature is 1130° C. which is a lower temperature than the sintering temperature of the NASICON which is the solid electrolyte by 50° C. or more, the YSZ layer is not sintered so that a lot of pores are present. FIG. 11 is an enlarged photograph of the YSZ layer as the reference electrode which is co-sintered at 1130° C. As illustrated in FIG. 11, it can be seen that a lot of pores are included in the YSZ layer. Further, when the co-sintering temperature is 1250° C., which is a higher temperature than the sintering temperature of the NASICON which is the solid electrolyte by 50° C. or more, there is a problem that the NASICON which is the solid electrolyte is dissolved. FIG. 12 is an enlarged photograph of the YSZ layer which is the reference electrode which is co-sintered at 1180° C. according to the exemplary embodiment of the present invention. As illustrated in FIG. 12, it can be seen that the NASICON which is the solid electrolyte is uniformly sintered by sintering the YSZ layer and the number of pores is reduced compared with FIG. 11.

In the present invention, the temperature of co-sintering may be from about 1250° C. to about 1350° C. (e.g., about 1250° C., 1252° C., 1254° C., 1256° C., 1258° C., 1260° C., 1262° C., 1264° C., 1266° C., 1268° C., 1270° C., 1272° C., 1274° C., 1276° C., 1278° C., 1280° C., 1282° C., 1284° C., 1286° C., 1288° C., 1290° C., 1292° C., 1294° C., 1296° C., 1298° C., 1300° C., 1302° C., 1304° C., 1306° C., 1308° C., 1310° C., 1312° C., 1314° C., 1316° C., 1318° C., 1320° C., 1322° C., 1324° C., 1326° C., 1328° C., 1330° C., 1332° C., 1334° C., 1336° C., 1338° C., 1340° C., 1342° C., 1344° C., 1346° C., 1348° C., or about 1350° C.). Similarly, when the co-sintering temperature is 1230° C. which is a lower temperature than the sintering temperature of the LLT which is the solid electrolyte by 50° C. or more, the YSZ layer is not sintered so that a lot of pores are present. Further, when the co-sintering temperature is 1350° C., which is a higher temperature than the sintering temperature of the LLT which is the solid electrolyte by 50° C. or more, there is a problem that the LLT which is the solid electrolyte is dissolved.

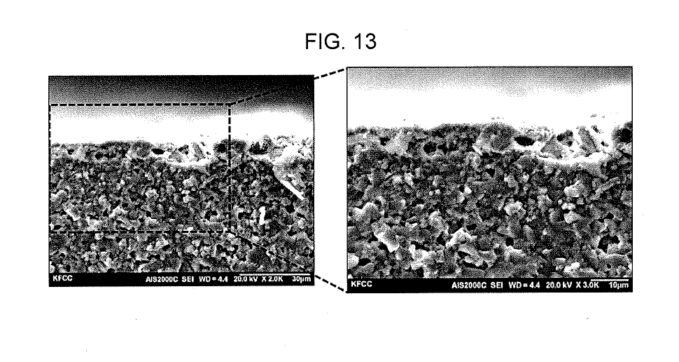

In the present invention, the co-sintering time may be at least 8 hrs to 24 hrs. If the co-sintering time is less than 8 hrs, the densification of the YSZ layer is not sufficiently performed. As a result, there is a problem in that the YSZ layer is not densely formed on the surface of the NASICON or the surface of the LLT which is the solid electrolyte and thus since gas flows in and out at the interface, the YSZ layer does not serve as a protective layer to influence the accuracy of the sensor. Furthermore, when the co-sintering time is 24 hrs or more, there is a problem that since the grain boundary is excessively grown, the risk of crack propagation is increased and the mechanical strength is deteriorated, so that the interface becomes unstable. FIG. 13 is an enlarged photograph of the YSZ layer as the reference electrode which is co-sintered for less than 8 hours. As illustrated in FIG. 13, it can be seen that when the co-sintering time is less than 8 hrs, the YSZ layer is not densely formed.

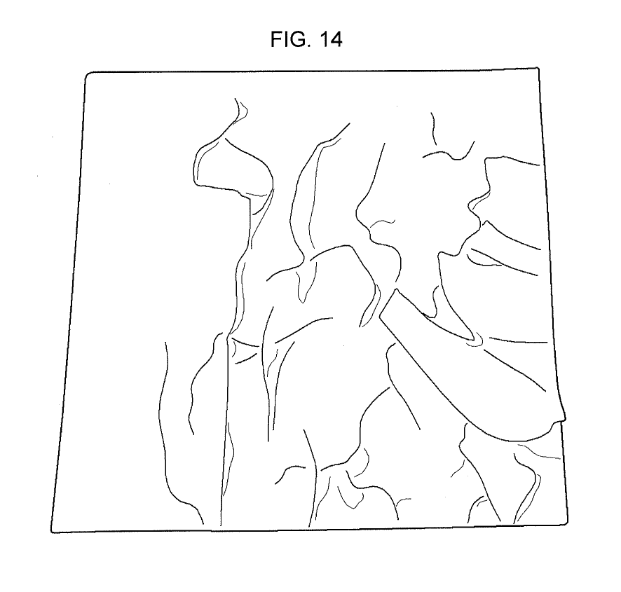

In the present invention, the yttria-stabilized zirconia (YSZ) is preferably coated with a thickness of more than 0 μm to 30 μm or less. The thickness of the YSZ is basically set to 10 μm or more which is a thickness to be printed when screen printing is performed once. When the coated thickness of the YSZ layer is more than 30 μm, the two layers are not bonded to each other due to a difference in shrinkage between the YSZ layer and the solid electrolyte to be separated and lifted. FIG. 14 is a photograph of the YSZ layer which is the reference electrode coated with a thickness of 50 μm. As illustrated in FIG. 14, it can be seen that the YSZ layer is lifted due to the difference in shrinkage. In addition, when the coated thickness of the YSZ layer is more than 30 μm, there is a problem in that the resistance of the sensor element increases. Accordingly, the coated thickness of the YSZ layer, that is, the thickness to be screen-printed, is preferably 30 μm or less, and it is preferred that the YSZ layer is uniformly coated on the surface of the solid electrolyte.

Meanwhile, it is preferred that the sensing electrode is any one of A₂CO₃(A=Li, Na), BCO₃(B═Ba, Ca, Sr) or a mixture thereof and the solid electrolyte is formed as a green sheet. In addition, the solid electrolyte is preferably NASICON, LLT, LISICON or UPON. In more detail, the solid electrolyte may be Na₁₊ₓZr₂Si_{X}P_{3-X}O₁₂ and 0<X<3, the solid electrolyte may be Li_{3X}La_{2/3-X}TiO₃ and 0.06<X<0.16, the solid electrolyte may be Li_{2+2X}Zn_{1-X}GeO₄ and 0<X<1, and the solid electrolyte may be Li₃PO_{4-X}N_{X} and 0.1<X<0.5. Further, the substrate may be made of alumina or mullite, and the method may further include a sealing step of sealing the side surface of the reference electrode with a sealant and the sealant is Na glass, Li glass, or a ceramic sealant.

According to the present invention, the method is applied to the solid electrolyte type gas sensor to fix the activity of the reference electrode, thereby inducing faster electrode stabilization and shortening the initial stabilization speed of the product. Further, in the case of sintering a material requiring high-temperature sintering such as YSZ and a material capable of low-temperature sintering together, the present invention has an advantage of obtaining a dense YSZ layer at a lower temperature by using shrinkage stress of a material sintered at a low temperature. Further, the present invention has an advantage of reducing production cost by omitting the heat-treating step for sintering for each layer. Furthermore, the present invention has an advantage of forming a denser boundary than other methods such as thermal bonding in the related art by applying to the double layer production.

Hereinafter, the present invention will be described in more detail through Examples. These Examples are just to exemplify the present invention, and it is apparent to those skilled in the art that it is not interpreted that the scope of the present invention is not limited to these Examples.

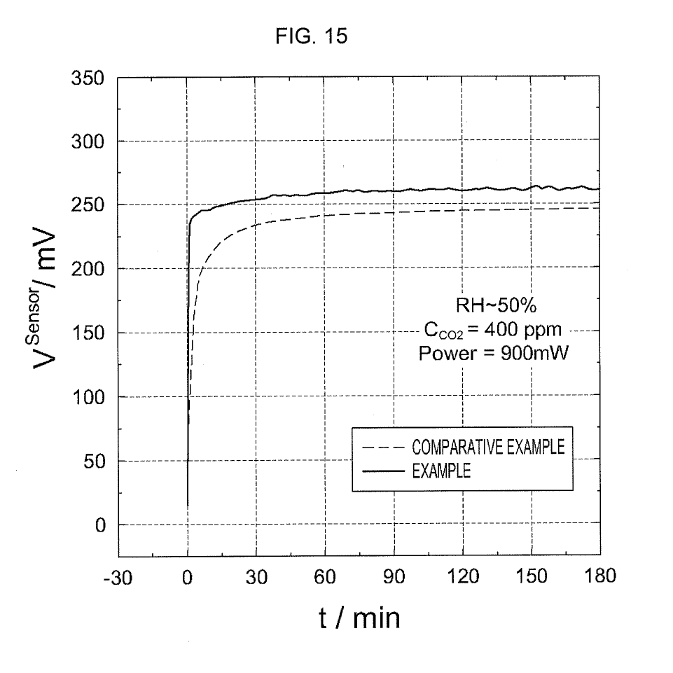

FIG. 15 is a graph illustrating a measured voltage with time according to an Example of the present invention and a Comparative Example as the related art in the case where a solid electrolyte is NASICON. In Comparative Example 1 as the related art, a sensing electrode including carbonate and Pt, a solid electrolyte as a NASICON, a reference electrode including Li—Ti—O and Pt, and a substrate are laminated in sequence, and in Example 1 of the present invention, a sensing electrode including carbonate and Pt, NASICON as a solid electrolyte, a YSZ layer as a reference electrode, and a substrate are laminated in sequence. Conditions of measuring the voltage are a relative humidity of 50%, a CO₂ concentration of 400 ppm, and power of 900 mW. A horizontal axis of the graph corresponds to time and a unit is minute. A vertical axis corresponds to the voltage of the sensor, and a unit corresponds to mV. When describing test results of Example 1 of the present invention and Comparative Example 1 of the related art measured under the above conditions, it can be seen that in Example 1, the voltage reaches a voltage corresponding to the equilibrium within a shorter time than Comparative Example 1.

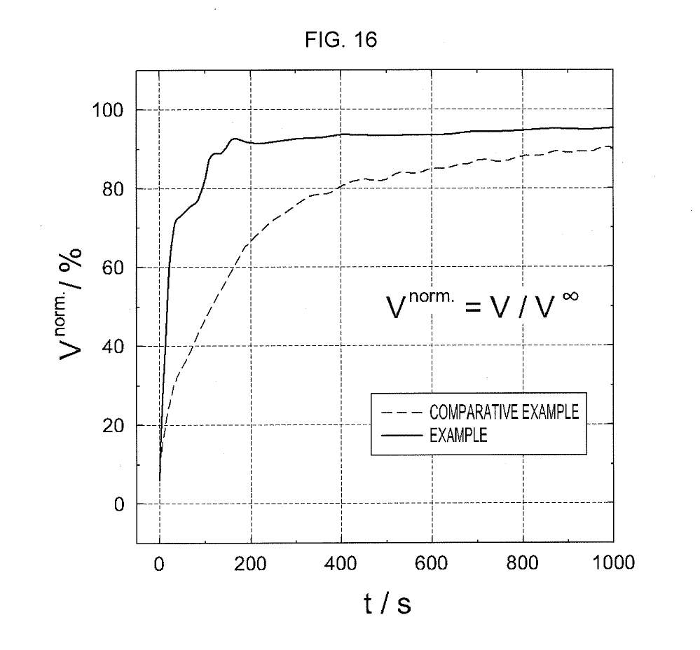

FIG. 16 is a graph illustrating normalizing of the measured voltage with time according to an Example of the present invention and a Comparative Example as the related art in the case where a solid electrolyte is NASICON. In Comparative Example 1 as the related art, a sensing electrode including carbonate and Pt, a solid electrolyte as a NASICON, a reference electrode including Li—Ti—O and Pt, and a substrate are laminated in sequence, and in Example 1 of the present invention, a sensing electrode including carbonate and Pt, NASICON as a solid electrolyte, a YSZ layer as a reference electrode, and a substrate are laminated in sequence. Conditions of measuring the voltage are a relative humidity of 50%, a CO₂ concentration of 400 ppm, and power of 900 mW. A horizontal axis of the graph corresponds to time and a unit is second. The vertical axis corresponds to a value obtained by dividing the measured voltage of the sensor by the equilibrium voltage and a unit corresponds to %. Like FIG. 15, in FIG. 16, it can be seen that in Example 1 of the present invention, the voltage reaches an equilibrium state within a shorter time than Comparative Example 1.

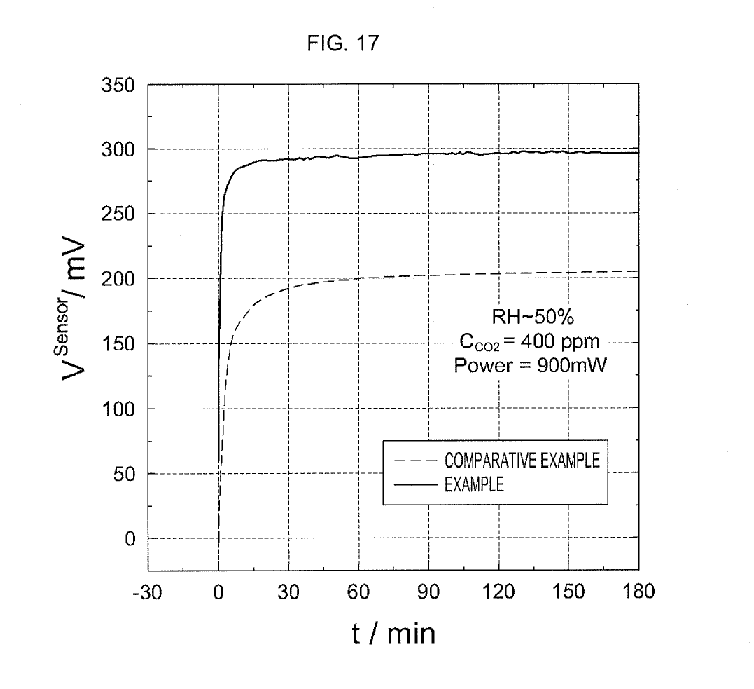

FIG. 17 is a graph illustrating a measured voltage with time according to an Example of the present invention and a Comparative Example as the related art in the case where a solid electrolyte is LLT. In Comparative Example 2 as the related art, a sensing electrode including carbonate and Pt, a solid electrolyte as a LLT, a reference electrode including Li—Ti—O and Pt, and a substrate are laminated in sequence, and in Example 2 of the present invention, a sensing electrode including carbonate and Pt, LLT as a solid electrolyte, a YSZ layer as a reference electrode, and a substrate are laminated in sequence. Conditions of measuring the voltage are a relative humidity of 50%, a CO₂ concentration of 400 ppm, and power of 900 mW. A horizontal axis of the graph corresponds to time and a unit is minute. The vertical axis corresponds to the voltage of the sensor, and the unit corresponds to mV. When describing test results of Example 2 of the present invention and Comparative Example 2 of the related art measured under the above conditions, it can be seen that in Example 2, the voltage reaches a voltage corresponding to the equilibrium within a shorter time than Comparative Example 2.

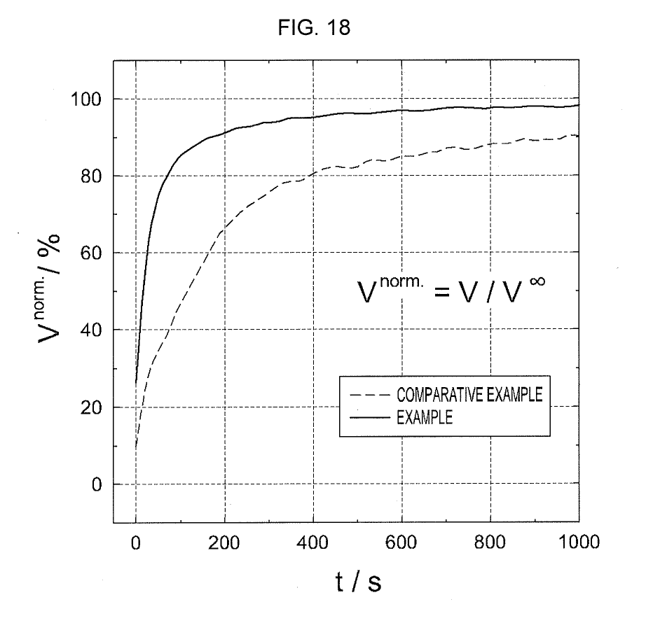

FIG. 18 is a graph illustrating normalizing of a measured voltage with time measured according to an Example of the present invention and a Comparative Example as the related art in the case where a solid electrolyte is LLT. In Comparative Example 2 as the related art, a sensing electrode including carbonate and Pt, a solid electrolyte as a LLT, a reference electrode including Li—Ti—O and Pt, and a substrate are laminated in sequence, and in Example 2 of the present invention, a sensing electrode including carbonate and Pt, LLT as a solid electrolyte, a YSZ layer as a reference electrode, and a substrate are laminated in sequence. Conditions of measuring the voltage are a relative humidity of 50%, a CO₂ concentration of 400 ppm, and power of 900 mW. A horizontal axis of the graph corresponds to time and a unit is second. The vertical axis corresponds to a value obtained by dividing the measured voltage of the sensor by the equilibrium voltage and a unit corresponds to %. Like FIG. 17, in FIG. 18, it can be seen that in Example 2 of the present invention, the voltage reaches an equilibrium state within a shorter time than Comparative Example 2.

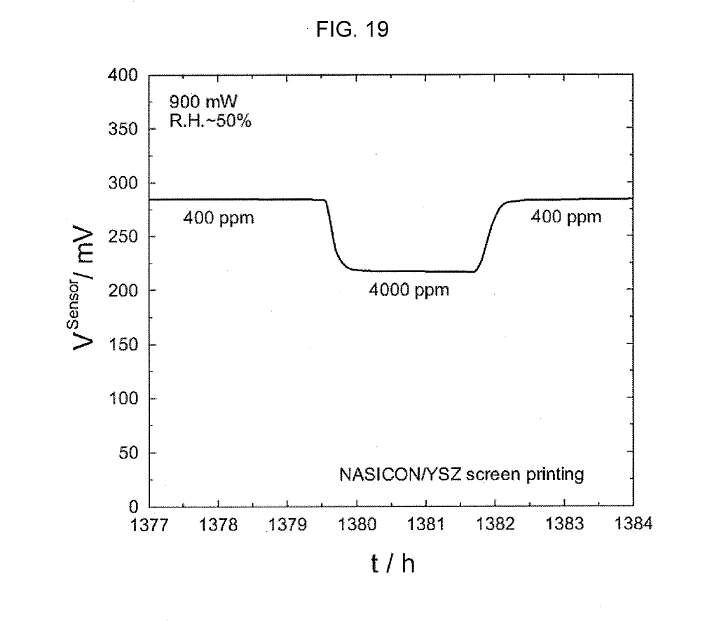

Meanwhile, in the present invention, the YSZ layer is densely formed at the interface of the solid electrolyte by co-sintering, thereby maintaining the same sensitivity as the related art, but improving the initial stabilization speed and accuracy. FIG. 19 is a graph illustrating a changed voltage with time in the case of changing a concentration of carbon dioxide according to an exemplary embodiment of the present invention. According to Example of the present invention, as a graph measured when the relative humidity is 50% at power of 900 mW, a horizontal axis represents time and a unit is hour. Further, a vertical axis corresponds to the voltage of the sensor, and a unit is mV. As illustrated in FIG. 19, the initial CO₂ concentration was changed from 400 ppm to 4000 ppm, and then changed to 400 ppm again. As a result, as illustrated in FIG. 19, it can be seen that a voltage which is precisely changed according to the CO₂ concentration is shown.

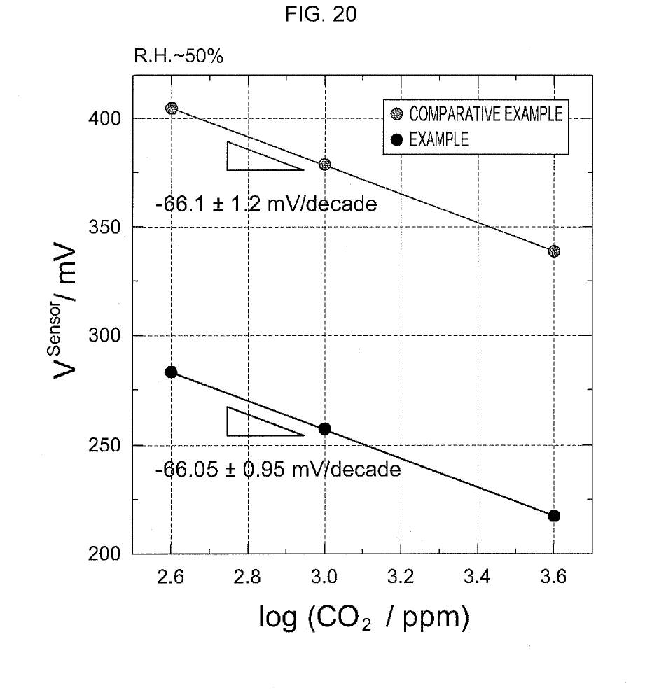

FIG. 20 is a graph illustrating changed voltage according to a concentration of carbon dioxide according to an exemplary embodiment of the present invention and a Comparative Example as the related art. As a graph measured when the condition for measuring the voltage was a relative humidity of 50%, a horizontal axis represents a log value of the CO₂ concentration and a vertical axis corresponds to the voltage of the sensor, and a unit is mV. When the sensitivity, which is a value of the corresponding voltage according to the changed CO₂ concentration as illustrated in FIG. 20, is −66.05±0.95 mV/decade in Example as compared with −66.1±1.2 mV/decade in Comparative Example, it can be seen that the sensitivity is not decreased and thus the same excellent performance is shown.

Therefore, the present invention includes the manufacturing method of bonding the solid electrolyte and the reference electrode through co-sintering, and thus there are advantages in that performance of the solid electrolyte CO₂ sensor is improved, the heat-heating step is removed so that the manufacturing cost is low, and the manufacturing time is reduced so that mass production is facilitated.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

What is claimed is:

1. A method for manufacturing a solid electrolyte type CO₂ sensor, the method comprising:
a bonding step of bonding a reference electrode on one surface of a solid electrolyte;
a first stacking step of stacking a sensing electrode on the other surface of the solid electrolyte facing the surface bonded to the reference electrode; and
a second stacking step of stacking a substrate on the other surface of the reference electrode facing the surface bonded to the solid electrolyte.

2. The method of claim 1, further comprising:
a coating step of coating a paste of the reference electrode on one surface of the solid electrolyte, before the bonding step.

3. The method of claim 2, wherein the coating step uses screen printing.

4. The method of claim 1, wherein in the bonding step, the solid electrolyte and the reference electrode are co-sintered.

5. The method of claim 4, wherein the co-sintering temperature is a temperature of from about 1150° C. to about 1250° C.

6. The method of claim 4, wherein the co-sintering temperature is a temperature of from about 1250° C. to about 1350° C.

7. The method of claim 4, wherein the co-sintering time is at least about 8 hrs to about 24 hrs.

8. The method of claim 2, wherein the paste of the reference electrode includes yttria-stabilized zirconia.

9. The method of claim 8, wherein the yttria-stabilized zirconia is coated with a thickness of more than 0 μm to about 30 μm or less.

10. The method of claim 1, wherein the sensing electrode is any one of A₂CO₃(A=Li, Na), BCO₃(B═Ba, Ca, Sr) or a mixture thereof.

11. The method of claim 1, wherein the solid electrolyte is formed as a green sheet.

12. The method of claim 5, wherein the solid electrolyte is Na_{1+X}Zr₂Si_{X}P_{3-X}O₁₂ and 0<X<3.

13. The method of claim 6, wherein the solid electrolyte is Li_{3X}La_{2/3-X}TiO₃ and 0.06<X<0.16.

14. The method of claim 6, wherein the solid electrolyte is Li_{2+2X}Zn_{1-X}GeO₄ and 0<X<1.

15. The method of claim 6, wherein the solid electrolyte is Li₃PO_{4-X}N_{X} and 0.1<X<0.5.

16. The method of claim 1, wherein the substrate is made of alumina or mullite.

17. The method of claim 1, further comprising:
sealing a side of the reference electrode with a sealant.

18. The method of claim 17, wherein the sealant is Na glass, Li glass, or a ceramic sealant.
